(19) United States
(12) Patent Application Publication
(10) Pub. No.: US 2006/0111288 A1
(43) Pub. Date: May 25, 2006

(54) Peripheral benzodiazepine receptor independent superoxide generation

(76) Inventors: Finbarr E. Cotter, Horsham (GB); Dean Anthony Fennell, Heler's Bay (IE)

Correspondence Address: Anthony C. Kuhlmann, Ph.D.;Brown Rudnick Berlack Israels LLP, One Financial Center, Boston, MA, 02111, US

(21) Appl. No.: 11/204,795

(22) Filed: Aug. 16, 2005

Related U.S. Application Data
(60) Provisional application No. 60/601,861, filed on Aug. 16, 2004.

(51) Int. Cl. A61K 38/17; A61K 31/02

(52) U.S. Cl. 514/12; 514/743

(57) ABSTRACT

A method of treating cancer through the application of a compound that causes the intra-mitochondrial generation of reactive oxygen species in tumor cells by a mechanism that is independent of the peripheral benzodiazepine receptor.

### CROSS-REFERENCE TO RELATED APPLICATIONS

This application claims benefit of U.S. Provisional application 60/601,861, filed Aug. 16, 2004.

### FIELD OF THE INVENTION

This invention relates to the field of generating superoxides within mitochondria such as through Complex I of the mitochondrial electron transport chain or NADPH oxidase for purposes of treating cancer and other diseases.

### BACKGROUND OF THE INVENTION

Selectively inducing apoptosis in tumor cells versus normal cells is an important goal of cancer therapeutic drug discovery. A number of drugs in clinical development are designed to selectively induce apoptosis, in particular by triggering signalling pathways in the cell that cause the generation of reactive oxygen species in the cytoplasm or at or near the cell membrane, whose end effect is to bring about the opening of the mitochondrial membrane permeability transition pore complex (PTPC). Opening the PTPC results in mitochondrial membrane depolarization, which causes the release of cytochrome c and initiates a programmed series of steps that lead to the death of the cell by apoptosis.

Induction of apoptosis by binding the peripheral benzodiazepine receptor (PBR) has received attention as a strategy for cancer therapeutics. The PBR is a mitochondrial protein with elusive function. It physically associates with the PTPC, the redox sensitive megachannel that dissipates the mitochondrial transmembrane potential, early during chemotherapy induced cell death. The PBR has been implicated in the regulation of the PTPC, on the basis of the cytotoxicity promoting activity of the isoquinoline carboxamide PK11195.

PK11195 exhibits nanomolar binding affinity to the PBR (1, 2). The PBR is an 18 kDa protein that localizes to the outer mitochondrial membrane in a pentameric configuration, as has been revealed by atomic force microscopy (3). The PBR is associated with the PTPC, whose mutimeric structure consists, on the outer mitochondrial membrane, of the voltage dependent anion channel (VDAC) and hexokinase, and on the inner mitochondrial membrane, of the adenine nucleotide translocator (ANT) and cyclophilin D(4-6). The PTPC in turn physically associates with both death agonist and death antagonist proteins of the Bcl-2 family that tune the apoptosis threshold of cells (7, 8). PK11195 has been shown to sensitize cells to a wide variety of apoptosis inducers in-vitro and in-vivo in a Bcl-2 and BCL-X_{L} resistant manner (9-12), implicating a PBR dependent effect on the PTPC (11). PK11195 has also been shown to mediate a diversity of cellular actions including inhibition of respiratory control (13), inhibition of cellular proliferation (14), and modulation of mitochondrial cholesterol translocation (15).

Although a role for the PBR has been implicated in mediating many of the cellular effects of PK11195, some pharmacology, such as inhibition of proliferation and enhancement of cytotoxicity, have been shown to occur exclusively in the micromolar range in vitro; orders of magnitude greater than that required to saturate the receptor (16, 17). Accordingly, it is our belief that the functions ascribed to PBR have been erroneously reported thereby frustrating attempts to capitalize on observed cellular effects to identify new therapeutic compounds especially useful for inter alia treating cancer.

It is an aspect of the present invention to disclose a new mechanism for explaining the effects of PK11195.

It is another aspect of the present invention to provide methods which utilize the newly disclosed mechanisms to screen for compounds capable of generating reactive oxygen species (ROS) in the proper location.

It is yet another aspect of the present invention to provide methods for treating cancer using reactive oxygen species.

### REFERENCES

1. Hirsch, J. D., Beyer, C. F., Malkowitz, L., Loullis, C. C., and Blume, A. J. Characterization of ligand binding to mitochondrial benzodiazepine receptors. Mol Pharmacol, 35: 164-172, 1989.
2. Anholt, R. R. H., Pedersen, P. L., De Souza, E. B., and Snyder, S. H. The peripheral-type benzodiazepine receptor. Localization to the mitochondrial outer membrane. J Biol Chem, 261: 576-583, 1986.
3. Papadopoulos, V., Boujrad, N., Ikonomovic, M. D., Ferrara, P., and Vidic, B. Topography of the Leydig cell mitochondrial peripheral-type benzodiazepine receptor. J Biol Chem, 269: 22105-22112, 1994.
4. Szabo, I. and Zoratti, M. The mitochondrial megachannel is the permeability transition pore. J Bioenerg Biomembr, 24: 111-117, 1992.
5. McEnery, M. W., Snowman, A. M., Trifiletti, R. R., and Snyder, S. H. Isolation of the mitochondrial benzodiazepine receptor: association with the voltage-dependent anion channel and the adenine nucleotide carrier. Proc Natl Acad Sci U S A, 89: 3170-3174, 1992.
6. McEnery, M. W. The mitochondrial benzodiazepine receptor: Evidence for association with the voltage-dependent anion channel (VDAC). J Bioenerg Biomembranes, 24: 63-69, 1992.
7. Narita, M., Shimizu, S., Ito, T., Chittenden, T., Lutz, R. J., Matsuda, H., and Tsujimoto, Y. Bax interacts with the permeability transition pore to induce permeability transition and cytochrome c release in isolated mitochondria. Proc Natl Acad Sci U S A, 95: 14681-14686, 1998.
8. Marzo, I., Brenner, C., Zamzami, N., Susin, S. A., Beutner, G., Brdiczka, D., Remy, R., Xie, Z. H., Reed, J. C., and Kroemer, G. The permeability transition pore complex: A target for apoptosis regulation by caspases and Bcl-2-related proteins. J EXP MED. Journal of Experimental Medicine, 187: 1261-1271, 1998.
9. Banker, D. E., Cooper, J. J., Fennell, D. A., Willman, C. L., Appelbaum, F. R., and Cotter, F. E. PK11195, a peripheral benzodiazepine receptor ligand, chemosensitizes acute myeloid leukemia cells to relevant therapeutic agents by more than one mechanism. Leuk Res, 26: 91-106, 2002.
10. Pastorino, J. G., Simbula, G., Yamamoto, K., Glascott, P. A., Jr., Rothman, R. J., and Farber, J. L. The cytotoxicity of tumor necrosis factor depends on induction of the mitochondrial permeability transition. J Biol Chem, 271: 29792-29798, 1996.
11. Hirsch, T., Decaudin, D., Susin, S. A., Marchetti, P., Larochette, N., Resche-Rigon, M., and Kroemer, G. PK11195, a ligand of the mitochondrial benzodiazepine receptor, facilitates the induction of apoptosis and reverses Bcl-2-mediated cytoprotection. Exp Cell Res, 241: 426-434, 1998.
12. Okaro, A. C., Fennell, D. A., Corbo, M., Davidson, B. R., and Cotter, F. E. PK11195, a mitochondrial benzodiazepine receptor antagonist, reduces apoptosis threshold in Bcl-X(L) and Mcl-1 expressing human cholangiocarcinoma cells. Gut, 51: 556-561, 2002.
13. Hirsch, J. D., Beyer, C. F., Malkowitz, L., Beer, B., and Blume, A. J. Mitochondrial benzodiazepine receptors mediate inhibition of mitochondrial respiratory control. Mol Pharmacol, 35: 157-163, 1989.
14. Landau, M., Weizman, A., Zoref-Shani, E., Beery, E., Wasseman, L., Landau, O., Gavish, M., Brenner, S., and Nordenberg, J. Antiproliferative and differentiating effects of benzodiazepine receptor ligands on B16 melanoma cells. Biochem Pharmacol, 56: 1029-1034, 1998.
15. Tsankova, V., Magistrelli, A., Cantoni, L., and Tacconi, M. T. Peripheral benzodiazepine receptor ligands in rat liver mitochondria: effect on cholesterol translocation. Eur J Pharmacol, 294: 601-607, 1995.
16. Zisterer, D. M., Gorman, A. M. C., Williams, D. C., and Murphy, M. P. The effects of the peripheral-type benzodiazepine acceptor ligands, Ro 5-4864 and PK 11195, on mitochondrial respiration. Methods Find Exp Clin Pharmacol, 14: 85-90, 1992.
17. Fennell, D. A., Corbo, M., Pallaska, A., and Cotter, F. E. Bcl-2 resistant mitochondrial toxicity mediated by the isoquinoline carboxamide PK11195 involves de novo generation of reactive oxygen species. Br J Cancer, 84: 1397-1404, 2001.
18. Carayon, P., Portier, M., Dussossoy, D., Bord, A., Petitpretre, G., Canat, X., Le Fur, G., and Casellas, P. Involvement of peripheral benzodiazepine receptors in the protection of hematopoietic cells against oxygen radical damage. Blood, 87:3170-3178, 1996.
19. Zisterer, D. M., Campiani, G., Nacci, V., and Williams, D. C. Pyrrolo-1,5-benzoxazepines induce apoptosis in HL-60, Jurkat, and Hut-78 cells: a new class of apoptotic agents. J Pharmacol Exp Ther, 293: 48-59, 2000.
20. Kozikowski, A. P., Kotoula, M., Ma, D., Boujrad, N., Tuckmantel, W., and Papadopoulos, V. Synthesis and biology of a 7-nitro-2,1,3-benzoxadiazol-4-yl derivative of 2-phenylindole-3-acetamide: a fluorescent probe for the peripheral-type benzodiazepine receptor. J Med Chem, 40: 2435-2439, 1997.
21. Gorman, A., McGowan, A., and Cotter, T. G. Role of peroxide and superoxide anion during tumour cell apoptosis. FEBS Lett, 404: 27-33, 1997.
22. Crompton, M. The mitochondrial permeability transition pore and its role in cell death. Biochem J, 341: 233-249, 1999.
23. Pastorino, J. G., Simbula, G., Gilfor, E., Hoek, J. B., and Farber, J. L. Protoporphyrin IX, an endogenous ligand of the peripheral benzodiazepine receptor, potentiates induction of the mitochondrial permeability transition and the killing of cultured hepatocytes by rotenone. J Biol Chem, 269: 31041-31046, 1994.
24. Larochette, N., Decaudin, D., Jacotot, E., Brenner, C., Marzo, I., Susin, S. A., Zamzami, N., Xie, Z., Reed, J., and Kroemer, G. Arsenite induces apoptosis via a direct effect on the mitochondrial permeability transition pore. Exp Cell Res, 249: 413-421, 1999.
25. Ravagnan, L., Marzo, I., Costantini, P., Susin, S. A., Zamzami, N., Petit, P. X., Hirsch, F., Goulbern, M., Poupon, M. F., Miccoli, L., Xie, Z., Reed, J. C., and Kroemer, G. Lonidamine triggers apoptosis via a direct, Bcl-2-inhibited effect on the mitochondrial permeability transition pore. Oncogene, 18: 2537-2546, 1999.
26. Camins, A., Diez-Fernandez, C., Camarasa, J., and Escubedo, E. Cell surface expression of heat shock proteins in dog neutrophils induced by mitochondrial benzodiazepine receptor ligands. Immunopharmacology, 29: 159-166, 1995.
27. Burkitt, M. J. and Wardman, P. Cytochrome C is a potent catalyst of dichlorofluorescin oxidation: implications for the role of reactive oxygen species in apoptosis. Biochem Biophys Res Commun, 282: 329-333, 2001.
28. Cai, J. and Jones, D. P. Superoxide in apoptosis. Mitochondrial generation triggered by Cytochrome C loss. J Biol Chem. Journal of Biological Chemistry, 273: 11401-11404, 1998.
29. Costantini, P., Chernyak, B. V., Petronilli, V., and Bernardi, P. Selective inhibition of the mitochondrial permeability transition pore at the oxidation-reduction sensitive dithiol by monobromobimane. FEBS Lett, 362: 239-242, 1995.
30. Petronilli, V., Costantini, P., Scorrano, L., Colonna, R., Passamonti, S., and Bernardi, P. The voltage sensor of the mitochondrial permeability transition pore is tuned by the oxidation-reduction state of vicinal thiols. Increase of the gating potential by oxidants and its reversal by reducing agents. J Biol Chem, 269: 16638-16642, 1994.
31. Chernyak, B. V. and Bernardi, P. The mitochondrial permeability transition pore is modulated by oxidative agents through both pyridine nucleotides and glutathione at two separate sites. Eur J Biochem, 238: 623-630, 1996.
32. Costantini, P., Belzacq, A. S., Vieira, H. L., Larochette, N., de Pablo, M. A., Zamzami, N., Susin, S. A., Brenner, C., and Kroemer, G. Oxidation of a critical thiol residue of the adenine nucleotide translocator enforces bcl-2-independent permeability transition pore opening and apoptosis. Oncogene, 19: 307-314, 2000.
33. Zamzami, N., Marzo, I., Susin, S. A., Brenner, C., Larochette, N., Marchetti, P., Reed, J., Kofler, R., and Kroemer, G. The thiol crosslinking agent diamide overcomes the apoptosis-inhibitory effect of Bcl-2 by enforcing mitochondrial permeability transition. Oncogene, 16: 1055-1063, 1998.
34. Chelli, B., Falleni, A., Salvetti, F., Gremigni, V., Lucacchini, A., and Martini, C.

Peripheral-type benzodiazepine receptor ligands: mitochondrial permeability transition induction in rat cardiac tissue. Biochem Pharmacol, 61: 695-705, 2001.
35. Decaudin, D., Geley, S., Hirsch, T., Castedo, M., Marchetti, P., Macho, A., Kofler, R., and Kroemer, G. Bcl-2 and Bcl-X_{L} antagonize the mitochondrial dysfunction preceding nuclear apoptosis induced by chemotherapeutic agents. Cancer Res, 57: 62-67, 1997.

### SUMMARY OF THE INVENTION

In accordance with the various aspects of the present invention and a new understanding of the operative intracellular mechanisms there are provided new methods for identifying reactive oxygen species which operate through the mitochondrial and which can generate cytotoxic effects especially useful for treating cancer.

We have discovered, that PK11195 induces mitochondrial depolarisation in HL60 human leukaemia cells in the micromolar concentration range, and that this induction of mitochondrial depolarization is inhibited by bongkrekic acid and involves permeability transition. PK11195 mediates catalase inhibitable, dose-dependent generation of hydrogen peroxide, localised to mitochondria in both PBR-positive BV173 and PBR-negative Jurkat leukaemia cells. The generation of superoxide (O^{2-·}) is required for mediating mitochondrial depolarisation, as evidenced by the inhibitory effect of the manganese O^{2-·} dismutase mimetic, Manganese (III) tetrakis (4-benzoic acid) porphyrin chloride (MnTBAP) on the kinetics of mitochondrial depolarisation. PK11195 has previously been shown to antagonize the anti-death activity of the mitochondrial proteins Bcl-2 and BCl-X_{L}. We have also discovered that this property results exclusively from the pro-oxidant activity of PK11195 on the redox sensitive PTPC, rather than via a PBR dependent interaction with the PTPC and megachannel formation as previously erroneously reported.

We have discovered that PK11195 generates reactive oxygen species in the micromolar range of concentration, causing mitochondrial toxicity via the PTPC with promotion of mitochondrial permeability transition (MPT). Furthermore, the expression of the PBR is not a prerequisite for this pro-oxidant activity implicating a direct action of the PK11195 molecule.

As a result, the present invention concerns a method for inducing apoptosis of cells (e.g., treating cancer) in a subject comprising administering to the subject a therapeutically effective amount of an agent for activating the Caspase 9 apoptosis pathway wherein the agent binds to mitochondria of the cells resulting in intra-mitochondrial superoxide generation leading to release of Cytochrome C within the cells and activation of the Caspase 9 apoptosis pathway. A preferred result occurs when the agent is internalized within the mitochondria, or when administration of the agent results in intra-mitochondrial superoxide generation by interaction of the agent with mitochondrial NADPH oxidase, or when administration of the agent results in intra-mitochondrial superoxide generation by enzymatic action of NADPH oxidase, most preferably by enzymatic action directly on the agent itself, and especially when the NADPH oxidase removes at least one halogen atom (e.g., F, Cl, Br, and I) from the agent. Such a halogen could be chlorine. Preferred embodiments involve removal by NADPH oxidase of at least one halogen atom from the agent and replacement of each such removed halogen atom with oxygen. In a preferred method of the present invention, the mitochondria have surface transition pores with an adenine nucleotide translocator portion and the generated superoxide causes thiol oxidation of the adenine nucleotide translocator portion of the mitochondrial surface transition pores. Preferred agents useful with the above methods include PK11195, MPTP, and analogs thereof. The therapeutic agent may be administered in one dose or multiple doses which may be spaced by about 24 hours, 48 hours, three days, one week, two weeks, four weeks, or more. In another embodiment, the method for inducing apoptosis further includes the administration of an anti-neoplastic agent. The anti-neoplastic agent may be administered in one dose or multiple doses which may be spaced by about 24 hours, 48 hours, three days, one week, two weeks, four weeks, or more. Further, the anti-neoplastic agent may be administered simultaneously with the therapeutic agent or at a different time (either prior or subsequent). In a preferred embodiment, the therapeutic agent is administered about 12 hours, 24 hours, 48 hours, or one week prior to at least one administration of the anti-neoplastic agent.

The present invention also provides a method for sensitizing cells to anti-cancer treatment comprising administering to the cells an agent for causing release of Cytochrome C from mitochondria within the cells and activation of the Caspase 9 apoptosis pathway. The agent may be administered either simultaneously or prior to administration of an anti-neoplastic agent.

The present invention also provides a method for identifying a compound useful for the treatment of a cancer, the method comprising the steps of: (a) providing a sample containing viable mitochondria; (b) contacting the sample with a candidate compound; and (c) assessing either the level of superoxide production by the mitochondria or the membrane potential of the mitochondria, wherein a compound that increases superoxide production or alters membrane potential is identified as a compound useful for the treatment of a cancer. Preferably, the viable mitochondria are provided in a mitoplast preparation or within viable cells. In one embodiment, the mitochondria do not express substantial amounts of the peripheral benzodiazepine receptor. In another embodiment, when the mitochondria are provided in viable cells, the cells in do not bind NBD FGIN-1-27. Useful cells include, for example, HL60 promyelocytic leukemia cells or Jurkat T cell leukemia cells. In another embodiment, superoxide production is detected using CMH2DCF fluorescence.

The present invention also provides a method for screening one or more agents for making a preliminary determination of which of the agents may be useful as an anti-cancer compound comprising contacting the agents to be screened with NADPH oxidase under conditions permitting an enzymatic reaction and identifying as desirable agents those agents which have had at least one or more halogen atoms removed or which have been transformed into a reactive oxygen species by action of the NADPH oxidase.

The present invention also provides a method for identifying a compound useful for the treatment of a cancer, the method comprises the steps of: (a) providing a sample containing NADPH oxidase; (b) contacting the sample with a candidate compound containing a halogen atom; and (c) assessing the removal of the halogen atom from the compound or the generation of reactive oxygen species in the sample, wherein a compound having a halogen atom removed by the NADPH oxidase or a compound causing the generation of reactive oxygen species is identified as a compound useful for the treatment of a cancer. Preferred agents are those identified by any of the foregoing screening methods.

The present invention also provides a method for identifying cancers, cancer cells, tumors or patients having such cancers or tumors which may be successfully treated with an agent which results in activation of apoptosis through a Caspase 9 pathway comprising identifying those cancers or tumors having a level of NADPH oxidase level sufficient to transform a therapeutically effective amount of an agent into a reactive oxygen species. Preferably, the cells are obtained from a human patient. Cells may be obtained using a biopsy.

The present invention also provides an agent for treating cancer or for use with other anti-cancer therapeutic compounds wherein the agent activates or binds to mitochondria in cells causing intra-mitochondrial superoxidase generation leading to release of Cytochrome C within the cells and activation of the Caspase 9 apoptosis pathway. A preferred agent interacts with NADPH oxidase resulting in formation of intra-mitochondrial superoxide. A more preferred agent further comprises a therapeutically acceptable formulation comprising a pharmaceutically acceptable carrier.

The present invention also provides a method for preferentially killing cancer cells over non-cancer cells in a mammal comprising separate or simultaneous co-administration with a chemotherapeutic compound to the mammal a therapeutically effective amount of a agent which increases the intra-mitochondrial generation of a reactive oxygen species. In a preferred embodiment, the intra-mitochondrial generation of a reactive oxygen species occurs via interaction of the agent with NADPH oxidase. In a still more preferred embodiment, the agent binds to mitochondria in cancer cells causing intra-mitochondrial superoxidase generation leading to activation of the Caspase 9 apoptosis pathway in the cancer cells. In a still more preferred embodiment, the method involves the intra-mitochondrial generation of a reactive oxygen species via interaction with NADPH oxidase.

The present invention also provides a method for inducing apoptosis of lymphocytes in a subject comprising administering to the subject a therapeutically effective amount of an agent for activating the Caspase 9 apoptosis pathway wherein the agent binds to mitochondria of the lymphocytes resulting in intra-mitochondrial superoxide generation leading to release of Cytochrome C within the lymphocytes and activation of the Caspase 9 apoptosis pathway. This method may be used to treat any inflammatory disease associated with lymphocyte activation including, for example, rheumatoid arthritis, lupus, and other auto-immune diseases.

Preferred agents which may be used in any of the foregoing methods of the invention include PK11195, MPTP, and analogs thereof.

### BRIEF DESCRIPTION OF THE DRAWINGS

Further understanding of the various principles and aspects of the present invention may be had by reference to the figures wherein:

FIG. 1 shows induction of MPT in HL60 leukaemia cells by PK11195. Synchronous mitochondrial depolarisation of HL60 mitochondria (1A) measured by the lipophilic cation DiOC₆(3) resulted in a left shift in median fluorescence intensity (1B) that is reversed by pre-treatment with the ANT specific ligand BA (1C). 1D. Calcein fluorescence corresponding to mitochondria with closed PTPCs (left) underwent quenching following PK11195 treatment (right), consistent with MPT and coinciding with reduction in DiOC₆(3) fluorescence.

FIG. 2 shows that mitochondria are the source of PK11195 mediated ROS. 2A. PK11195 produced a concentration dependent increase in hydrogen peroxide measured by CMH2DCF fluorescence in HL60 cells; this occurred in the micromolar concentration range. 2B. PK11195 induced hydrogen peroxide was prevented in HL60 cells preincubated with catalase. 2C. PK11195 produced a punctate cytoplasmic distribution of CMH2DCF fluorescence consistent with a mitochondrial location.

FIG. 3 shows that the peripheral benzodiazepine receptor is not required for PK11195 mediated hydrogen peroxide generation. 3A. Jurkat cells failed to demonstrate a punctate distribution of PBR stained by NBD FGIN 1 27 analogue, compared with BV173 leukaemia cells (3B). 3C. Expression of the PBR was not seen in Jurkat cells by RT PCR but can be demonstrated in BV173 cells consistent with NBD FGIN 1 27 fluorescence microscopy. 3C and 3D. PK11195 mediated an increase in CMH₂DCF fluorescence in both Jurkat cells and BV173 cells irrespective of the expression of PBR.

FIG. 4 shows O₂^{-·} generation by PK11195 directly induces mitochondrial depolarization in leukaemia cells. 4A. Ethidium fluorescence was increased by PK11195 compared with control. 4B. PK1115 induced increase in ethidium fluorescence was inhibited by the manganese O₂^{-·} dismutase mimetic, MnTBAP. 4C. The rate of mitochondrial depolarisation induced by PK11195 in HL60 leukaemia cells measured using DiOC₆(3) fluorescence, was reduced by MnTBAP.

FIG. 5 shows that the compound 1-methyl-4-phenyl-1,2,3,6-tefrahydropyridine (MPTP), like PK11195, induces killing of tumor cells by ROS, but that (HA14-1) does not. DoHH2 cells, a lymphoma cell line, were treated with MPTP, PK11195 and HA14-1 in the presence and absence of MnTBAP, a scavenger of ROS. The rate of mitochondrial depolarisation induced by PK11195 in HL60 leukaemia cells measured was using DiOC₆(3) fluorescence. MnTBAP inhibited MPTP-induced and PK11195-induced apoptosis, but not apoptosis induced by HA14-1. The three columns in each lane represent three time courses (L to R: 24 hr, 48 hr and 72 hr). The control lane (CON) shows apoptosis levels at 24, 48 and 72 hrs in untreated cells. The induction of apoptosis at the same time points as a result of treatment with 1000 micromolar MPTP is shown in lane “1000”. The “1000+MNT” shows the reduction in apoptosis induction following treatment with the superoxide scavenger MnTBAP.

The blockade of MPTP induction of apoptosis by MnTBAP parallels that of PK11195. The lane labelled “PK100” shows treatment with 100 micromolar PK11195, which is blocked by MnTBAP in the subsequent lane (“PK+MNT”). By contrast, however, the induction of apoptosis by 50 micromolar anti-tumor agent HA14-1, a compound that induces apoptosis via the mitochondrial transition pore but does not involve the NADPH oxidase pathway, is not blocked by MnTBAP (compare “HA50” and HA+MNT”).

### DETAILED DESCRIPTION OF THE INVENTION

Reagents.

Calcein-AM and 3-3′-dihexyloxacarbocyanine iodide (DiOC₆(3)), and chloromethyl-X-rosamine were purchased from Molecular Probes/Cambridge Bioscience, UK. Bongkrekic acid (BA) was purchased from Biomol (UK). 7 nitro 2,1,3, benoxadiazol-4-yl 2-phenylindole-3-acetamide (NBD FGIN-1-27 analogue) was purchased from Alexis biochemicals, Cambridge (UK). 1-(2-chlorophenyl)-N-methyl-N-methyl-N-(1-methylpropyl)-isoquinoline carboxamide (PK11195), propidium iodide, catalase, dihydroethidium, and all cell culture reagents were purchased from Sigma-Aldrich Ltd, UK. Manganese (III) tetrakis (4-benzoic acid) porphyrin chloride (MnTBAP) was purchased from Oxis Health products.

Cell Culture and Treatments.

HL60 promyelocytic leukaemia, Jurkat T cell leukaemia cells lacking the PBR,(18) (19) (kindly provided by Dr D. E. Banker and Dr F. Applebaum, The Fred Hutchinson Cancer Research Center, USA), and BV173 leukaemia cells were maintained in exponential suspension cultures in PPMI 1640 medium supplemented with 10% fetal calf serum, 5 mM glutamine, 100 μg/ml streptomycin and 100 U/ml penicillin. Cells were grown in a humidified atmosphere of 5% CO₂/95% air at 37° C. PK11195 was dissolved in ethanol at a stock concentration of 8.7 mg/ml, and added to cells at a 75 μM final concentration for 4 hours; vehicle alone was also used in medium.

Flow Cytometry and Fluorescence Microscopy

A Becton Dickinson FACScan (Oxford) was used to acquire 10,000 events using forward and scatter detectors with logarithmic amplification. Lymphoid enriched gates were defined. Corresponding DiOC₆(3) or CMH₂DCFDA fluorescence was analysed using the FL1 (530 nm) band pass filter. Propidium iodide or ethidium fluorescence were analysed using the FL3 (620 nm) band pass filter. List mode Data was analysed using WINMDI 2.8. Fluorescence microscopy was performed using a Zeiss Axioskop four colour florescence microscope with digital capture via a computer running IPLab Spectrum software.

Measurement of Mitochondrial Membrane Potential Depolarisation and MPT.

Cells were incubated for 20 minutes at 37° C. in the dark with the cationic lipophillic (amphipathic) probe DiOC₆(3) (80 nM), then counter stained for 10 minutes with propidium iodide (20 μg/ml). DiOC₆(3) is sequestered within the mitochondrial matrix due to the inner membrane potential (ΔΨₘ), according to the Nernst equation, dissipation leading to a reduction in mitochondrial retention and decreased cellular DiOC₆(3) fluorescence. Collapse of ΔΨₘ was prevented by incubating cells for 30 minutes with 50 μM bongkrekic acid. Events with increased propidium iodide fluorescence were subtracted, by gating, from the DiOC₆(3) histograms to eliminate dead cells, with loss of plasma membrane integrity. Dose-response curves used the calculating the proportion of DiOC₆(3) _{low} cells as the dependent variable on the ordinate. To measure MPT directly, cells were incubated with 1 μM calcein AM for 30 minutes, followed by 1 mM calcium cobalt. Cobalt quenches calcein fluorescence, but cannot traverse an intact inner mitochondrial membrane to enter the mitochondrial matrix, to which calcein equilibrates. Opening of the PTPC allows cobalt to enter, reducing calcein fluorescence.

Detection of PBR mRNA by Reverse Transcription-Polymerase Chain Reaction and Fluorescence Microscopy

Poly A+ mRNA was extracted from BV173 and Jurkat leukaemia cells using Quickprep (Pharmacia), and complementary DNA synthesized by single strand synthesis using Superscript II reverse transcriptase with degenerate primers. TAQ polymerase chain reaction was used to amplify a 590 base fragment of the PBR spanning exons 1-4 using the forward primer 5 CTAACTCCTGCCAGGCAGT (SEQ. ID NO.: 1) and the Reverse primer 5° CCATGTTC-CAAGAACATGC (SEQ. ID NO.: 2). Parallel amplification of retinoblastoma mRNA was used as a control amplicon. The peripheral benzodiazepine receptor was visualized by incubating Jurkat or BV173 cells with 1 μM NBD FGFN-1-27 analogue (20) for 45 minutes at 37° in the dark, visualized by fluorescence microscopy using the green wavelength, band pass filter.

Measurement and Inhibition of Hydrogen Peroxide and O₂^{-·} Generation

O₂^{-·} was detected by incubating cells for 15 minutes in 5 μM dihydroethidium, which is oxidized to ethidium. To test the effect of O₂^{-·} dismutase inhibitor on O₂^{-·} generation, cells were treated with 100 μM MnTBAP for 45 minutes. Hydrogen peroxide was detected by loading cells for 30 minutes with 5 μM CM-H₂DCFDA, and its formation inhibited by preincubation with 500 U/ml catalase for 30 minutes (21). Dose-response curves for CM-H₂DCFDA used the calculated proportion of CM-H₂DCFDA _{high} cells as the dependent variable.

### EXAMPLE 1

PK11195 Directly Induces BA Inhibitable MPT in HL60 Leukemic Cells.

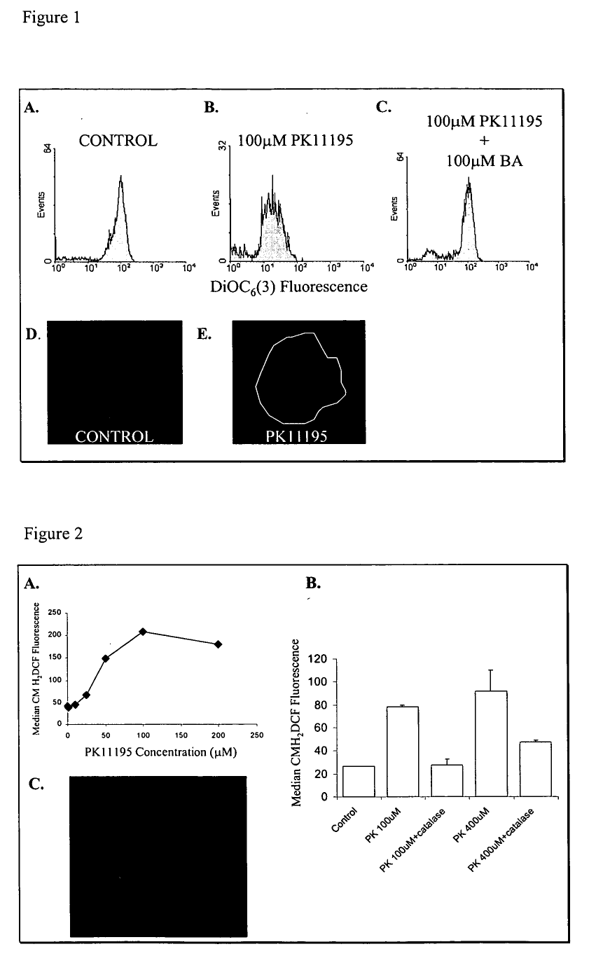

HL60 leukaemia cells treated with PK11195 exhibited a reduction in DiOC₆(3) fluorescence (FIG. 1B) detectable within 3 hours compared with control (FIG. 1A), consistent with collapse of mitochondrial ΔΨₘ. The ANT specific inhibitor BA prevented PK11195 mediated mitochondrial depolarisation (FIG. 1C), implicating the ANT in the process of PK11195 induced reduction in DiOC₆(3) fluorescence. Mitochondrial calcein fluorescence was quenched by PK11195 consistent with mitochondrial equilibration with cytosolic cobalt via open PTPCs (FIGS. 1D and 1E). Calcein quenching was not observed in BA treated cells (not shown).

### EXAMPLE 2

Dose Dependent Hydrogen Peroxide Generation Mediated by PK11195 is Localised to Mitochondria.

CMH₂DCF fluorescence in HL60 cells increased in a PK11195 concentration dependent manner, occurring in a 50-100 micromolar range of concentrations (FIG. 1A), consistent with the generation of ROS (FIG. 2A). This increase in CMH₂DCF fluorescence was inhibited in cells treated with catalase consistent with hydrogen peroxide dependent oxidation mediated by PK11195 (FIG. 2B). Fluorescence microscopy of CMH₂DCFDA loaded HL60 cells demonstrated a punctate cytoplasmic distribution of H₂0₂ generation following PK11195 treatment (FIG. 2C); this co-localised with that of the potentiometric probe CMX-Rosamine, consistent with mitochondrial generation of H₂0₂.

### EXAMPLE 3

The PBR is not Involved in PK11195 Induced H₂0₂ Generation.

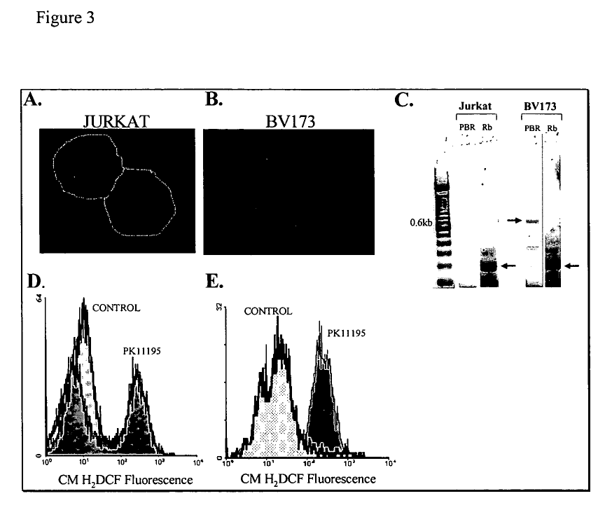

To determine the involvement of the PBR in PK11195 mediated ROS, generation of H₂0₂ was investigated in cell lines with differential PBR expression. The Jurkat T cell leukaemia line has previously been shown to be devoid of PBR expression. This was demonstrated via the absence of NBD FGIN-1-27 analogue binding observed by fluorescence microscopy (FIG. 3A). In contrast, BV173 leukaemia cells exhibited strongly positive staining of NBD FGIN-1-27 analogue, with a distinct punctate cytoplasmic distribution (FIG. 3B). Consistent with these findings, expression of the PBR was identified by RT-PCR in BV173 cells but not Jurkat cells (FIG. 3C). Irrespective of PBR expression however, PK11195 treatment produced an increase in CMH₂DCF fluorescence in both BV173 and Jurkat cells consistent with generation of H₂0₂ (FIGS. 3D and 3E).

### EXAMPLE 4

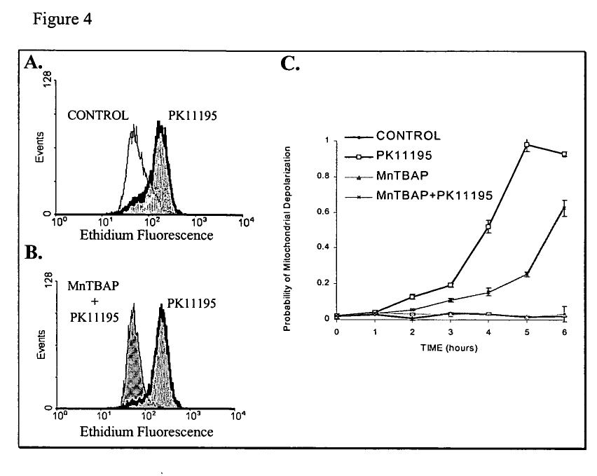

Mitochondrial toxicity mediated by PK11195 requires generation of O₂^{-·} can be physiologically dismutated to H₂0₂ by endogenous O₂^{-·} dismutases. To determine whether or not PK 11195 induced the generation of O₂^{-·} upstream of H₂0₂, ethidium fluorescence was measured following PK11195 treatment. An early increase in ethidium fluorescence was observed (FIG. 4A) that was inhibited by the manganese O₂^{-·} dismutase mimetic, MnTBAP (FIG. 4B). To determine the role of O₂^{-·} on mitochondrial depolarisation, the rate of reduction of DiOC₆(3) fluorescence was measured following PK11195 administration in the presence and absence of MnTBAP (FIG. 4C). Reduction in the rate of inner mitochondrial membrane depolarisation occurred in the presence of MnTBAP, consistent with a direct effect of PK11195 generated O₂^{-·} on the stability of the inner membrane potential, ΔΨₘ.

### EXAMPLE 5

Tumor Cell Killing by MPTP and PK11195, but not by HA14-1, is Blocked by the ROS Scavenger, MnTBAP.

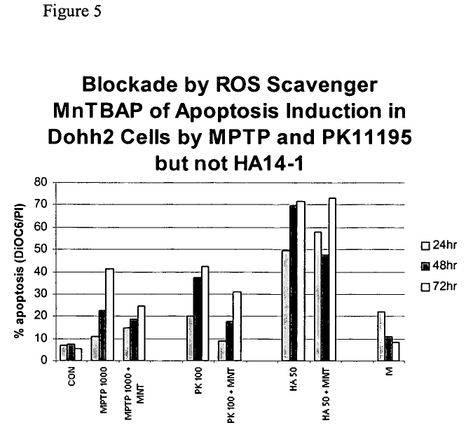

The above processes were used as a method for screening compounds for purposes of seeing whether such could be used to identify another compound capable of causing the formation of apoptosis inducing ROS. As a result of these efforts, it was discovered that MPTP also acts pursuant to the newly described mechanism of the present invention and can also serve as an anti-cancer therapeutic agent (FIG. 5). This was confirmed in part by the blocking effect of MnTBAP.

The induction of apoptosis by 50 micromolar anti-tumor agent HA14-1, a compound that induces apoptosis via the mitochondrial transition pore but does not involve the NADPH oxidase pathway, is not blocked by MnTBAP (compare “HA50” and HA+MNT”).

DoHH2 cells, a lymphoma cell line with high levels of the pro-apoptotic protein Bcl-2 due to a translocation between chromosome 14 and 18 (where the Bcl-2 gene is located), were exposed to MPTP, PK11195 and HA 14-1 in the presence and absence of the mitochondrial ROS scavenger, MnTBAP. MnTBAP inhibited MPTP-induced and PK11195-induced apoptosis, indicating that MPTP and PK11195 both act via intra-mitochondrial generation of ROS. but not apoptosis induced by HA14-1.

In addition, we discovered that PK11195 efficacy depends on NADPH oxidase levels. A PK11195 resistant lymphoblastic cell line was generated and the PK11195 sensitive and resistant cell line compared by gene expression array data. Lymphoblastic cell lines that were generated to be resistant to PK11195 express reduced levels of NADPH compared to lymphoblasts that are susceptible to PK11195. Moreover, PK11195 treatment induces apoptosis in primary chronic lymphatic leukaemia cells, which have higher levels of NADPH oxidase than normal cells, but does not do so in normal, non-malignant lymphocytes. Using gene expression arrays, we have discovered that NADPH oxidase is up-regulated in cell types that are sensitive to PK11195. Up-regulation of NADPH oxidase was confirmed by PCR. We suggest, while not wishing to be bound by such theory, that the lower levels of NADPH oxidase in normal cells, compared to tumor cells, renders them more resistant to induction of apoptosis by PK11195, whereas the higher levels of NADPH oxidase in tumor cells renders them more susceptible to induction of apoptosis by PK11195.

We investigated the mechanism by which PK11195 generates ROS. NMR studies performed on PK11195 treated cells showed that the Chlorine atom that is attached to PK11195 was cleaved from the PK11195 in the malignant (PK11195-sensitive) cells. It was also found that the chlorine was replaced with an atom of oxygen. As this reaction is one that can be carried out enzymatically by NADPH oxidase, we suggest, without wishing to be bound by such theory, that PK11195 generates ROS through the enzymatic action of NADPH oxidase.

### DISCUSSION

The PTPC plays a central role in the physiology of cell death and apoptosis (22). Facilitation of cell death by PK11195 of a variety of toxins has implicated the PBR in the regulation of cell death, acting as a putative modulator of PTPC function (10, 11, 23-25). We discovered however, that the PBR ligand PK11195 displays intracellular pro-oxidant activity that targets the PTPC via generation of O^{2-·} ROS production occurs independently of PBR expression, as demonstrated in PBR negative Jurkat T cells, and to an equal degree in PBR positive cells. The origin of H₂0₂ is mitochondrial, occurring at micromolar concentrations of PK11195. This is orders of magnitude greater than the PBR binding affinity of PK11195, and in the concentration range required to observe cytotoxic effects in sensitive cell lines such as HL60.

PK11195 has previously been shown to induce dose dependent expression of heat shock proteins HSP 72 and HSP 90 in canine neutrophils in the micromolar range of concentrations, a phenomenon suggested to be a consequence of oxidative stress (26). PK11195 induces ROS in the presence of an intact ΔΨₘ (17). Although cytochrome c is released from mitochondria by PK11195 (11), and has been shown to potently oxidize H₂DCF (27), failure of BCL-2 hyper-expression to modify ROS generation despite conferring apoptosis resistance (17) , and the inhibition of PK11195 induced H₂0₂ and O₂^{-·} by catalase and MnTBAP respectively, suggests a direct involvement of ROS. Release of cytochrome C results in a change from 4-electron to 1-electron reduction of O₂, and generation of O₂^{-·} (28), however, PK11195 is a potent inducer of ROS in ρ⁰ cells, devoid of a functional electron transport (17), strongly supporting a direct pro-oxidant activity.

The PTPC is a redox sensitive, multimeric protein complex with critical vicinal thiols at the matrix facing side of the ANT that regulate gating (29-31). Oxidation of cysteine 56 increases the probability of channel formation by the ANT (acting as the redox sensor), and underlies the cytotoxic activity of other pro-oxidants including diamide and ter-butylhydroperoxide (32, 33). Induction of mitochondrial depolarisation by PK11195 was inhibited by the ANT-specific ligand, bongkrekic acid, implicating MPT. The PTPC specific inhibitor of MPT, cyclosporin A has previously been shown to block PK11195 induced cardiac myocyte mitochondria swelling by cyclosporin A (34).

Using MnTBap to scavenge the superoxide has a similar effect to blocking the NADPH oxidase pathway. Only where NADPH oxidase has been functional will there be superoxide in the mitochondria to induce mitochondrial membrane depolarization. Thus MnTBAP does not reduce the membrane depolarization caused by MnTBAP, as HA14-1 does not work through the NADPH oxidase pathway, whereas it does reduce the membrane depolarization caused by PK11195 and MPTP. This confirms that MPTP is functioning through the NADPH oxidase pathway in a similar way to PK11195.

Anti-apoptotic proteins of the Bcl-2 family localize to the PTPC and are implicated in resistance to cytotoxic chemotherapy (35). Due to the ability of PK11195 to facilitate cell death in a Bcl-2 resistant manner, in common with pre-oxidants such as diamide, we suggest, without being bound to such theory, that alteration of mitochondrial redox state underlies this phenomenon rather than an allosteric effect of the PBR on the PTPC. Furthermore, the redox modifying activity of PK11195 may account for some of the diverse effects occurring in the micromolar range, that have previously been attributed exclusively to the PBR.

What is claimed is:

1. A method for inducing apoptosis of cells in a subject comprising administering to said subject a therapeutically effective amount of an agent for activating the Caspase 9 apoptosis pathway wherein said agent interacts with mitochondria of said cells resulting in intra-mitochondrial superoxide generation.

2. The method of claim 1, wherein said agent further induces the release of Cytochrome C within said cells.

3. The method of claim 1, wherein said agent is internalized within the mitochondria.

4. The method of claim 1, wherein said intra-mitochondrial superoxide generation is a result of an interaction between NADPH oxidase and said agent.

5. The method of claim 1, wherein said intra-mitochondrial superoxide generation is a result of enzymatic action of NADPH oxidase on said agent.

6. The method of claim 5, wherein said NADPH oxidase removes at least one halogen atom from said agent.

7. The method of claim 6, wherein said halogen is chlorine.

8. The method of claim 5, wherein said NADPH oxidase removes at least one halogen atom from said agent and replaces each of said removed halogen atoms with an oxygen atom.

9. The method of claim 1, wherein said mitochondria have surface transition pores with an adenine nucleotide translocator portion and said generated superoxide causes thiol oxidation of said adenine nucleotide translocator portion of said mitochondrial surface transition pores.

10. The method of claim 1, wherein said agent is selected from the group consisting of PK11195 and MPTP.

11. A method for treating a patient with cancer comprising administering to said patient a therapeutically effective amount of an agent for generating intra-mitochondrial superoxide.

12. The method of claim 11, wherein said agent further activates the Caspase 9 apoptosis pathway.

13. The method of claim 12, wherein said agent further induces the release of Cytochrome C within said cells.

14. The method of claim 11, wherein said agent is administered to said patient as a plurality of doses.

15. The method of claim 12, wherein said method further comprises administering an anti-neoplastic agent.

16. The method of claim 15, wherein at least one dose of said anti-neoplastic agent is administered within seven days of at least one dose of said agent.

17. The method of claim 15, wherein said anti-neoplastic agent is administered within 48 hours of at least one dose of said agent.

18. The method of claim 11 wherein said agent is selected from the group consisting of PK11195 and MPTP.

19. A method for sensitizing cells to anti-neoplastic treatment comprising administering to said cells an agent for activating the Caspase 9 apoptosis pathway wherein said agent interacts with mitochondria of said cells resulting in intra-mitochondrial superoxide generation.

20. The method of claim 19, wherein said agent further induces the release of Cytochrome C in said cells.

21. The method of claim 19, wherein said agent is administered prior to or simultaneously with an anti-neoplastic agent.

22. The method of claim 21, wherein said agent is administered within 48 hours prior to administration of an anti-neoplastic agent.

23. A method for identifying a compound useful for the treatment of a cancer, said method comprising the steps of:
a. providing a sample comprising viable mitochondria;
b. contacting said sample with a candidate compound; and
c. assessing the level of superoxide production by said mitochondria or the membrane potential of said mitochondria, wherein a compound that increases superoxide production or alters the membrane potential of said mitochondria is identified as a compound useful for the treatment of a cancer.

24. The method of claim 23, wherein said sample comprises mitoplasts.

25. The method of claim 23, wherein said sample comprises viable cells.

26. The method of claim 23, wherein said mitochondria do not comprise substantial amounts of the peripheral benzodiazepine receptor.

27. The method of claim 25 wherein said cells in said contacting step do not bind NBD FGIN-1-27.

28. The method of claim 25 wherein said cells in said contacting step are HL60 promyelocytic leukemia cells or Jurkat T cell leukemia cells.

29. The method of claim 23 wherein said identifying step comprises detecting CMH2DCF fluorescence.

30. A method for identifying a compound useful for the treatment of a cancer, said method comprising the steps of:
a. providing a sample comprising NADPH oxidase;
b. contacting said sample with a candidate compound comprising a halogen atom; and
c. assessing the removal of said halogen atom from said compound or the generation of reactive oxygen species in said sample, wherein a compound having a halogen atom removed by said NADPH oxidase or a compound causing the generation of reactive oxygen species is identified as a compound useful for the treatment of a cancer.

31. A method for identifying cancer cells that may be treated using an agent that results in activation of apoptosis through a Caspase 9 pathway comprising assessing the NADPH oxidase level in said cells, wherein cells possessing sufficient NADPH oxidase levels to transform a therapeutically effective amount of said agent into a reactive oxygen species are identified as cancer cells that may be treated using said agent.

32. The method of claim 31, wherein said cells are obtained from a human patient.

33. A agent for treating cancer or for use with other anti-cancer therapeutic compounds wherein said agent activates or binds to mitochondria in cells causing intra-mitochondrial superoxidase generation leading to release of Cytochrome C within said cells and activation of the Caspase 9 apoptosis pathway.

34. The agent of claim 33, wherein said agent interacts with NADPH oxidase resulting in formation of intra-mitochondrial superoxide.

35. A composition comprising the agent of claim 33 and a pharmaceutically acceptable carrier.

36. A method for inducing apoptosis of lymphocytes in a subject comprising administering to said subject a therapeutically effective amount of an agent for activating the Caspase 9 apoptosis pathway wherein said agent interacts with mitochondria of said lymphocytes resulting in intra-mitochondrial superoxide generation.

37. The method of claim 36, wherein said agent further induces the release of Cytochrome C within said lymphocytes.

38. The method of claim 36, wherein said agent is PK11195 or analogs thereof.

39. The method of claim 36, wherein said agent is MPTP or analogs thereof.
